(19) United States
(12) Patent Application Publication
(10) Pub. No.: US 2019/0289810 A1
(43) Pub. Date: Sep. 26, 2019

(54) SMART DEFECATION PLATE INCLUDING COMPANION ANIMAL MEDICAL EXAMINATION KIT

(71) Applicant: SQUARE CO., LTD., Daejeon (KR)

(72) Inventor: Jeho PARK, Daejeon (KR)

(21) Appl. No.: 16/318,404

(22) PCT Filed: Nov. 15, 2016
(86) PCT No.: PCT/KR2016/013155

§ 371 Date: Jan. 17, 2019

(30) Foreign Application Priority Data
Jul. 21, 2016 (KR) 10-2016-0092631

(51) Int. Cl. A01K 1/01; A01K 23/00

(52) U.S. Cl. CPC A01K 1/0107; A01K 29/00; A01K 23/00

(57) ABSTRACT

The present invention relates to a smart defecation plate including a companion animal medical examination kit. More specifically, the smart defecation plate includes: a body the center of which is formed in a concave shape; an upper plate which is coupled to the top of the body, and in which defecation holes are formed in a grid pattern; a pad part which includes a waterproof pad and absorption pads attached to the waterproof pad and configured to absorb a fluid entering through the defecation holes and which is disposed between the body and the upper plate; and a medical examination kit which is disposed on the top of the pad part. According to the present invention, the medical examination kit is provided on the top of the pad part, and thus the health state of a companion animal may be diagnosed through an introduced fluid.

### TECHNICAL FIELD

The present invention relates to a smart defecation plate including a companion animal medical examination kit.

More specifically, the present invention relates to a smart defecation plate including a companion animal medical examination kit, in which a medical examination kit is provided on the top of a pad part, thereby enabling the health state of a companion animal to be diagnosed using a fluid that is introduced into the pad part.

### BACKGROUND ART

Recently, the relationship between humans and companion animals has been gradually redefined as the relationship between friends, peers, or companions, other than the relationship between masters and servants. There is a tendency for the number of people, who consider their companion animals to be companions or family members of their life and then adopt companion animals in order to live their lives together with them, rather than raising their companion animals for hobby or fun in the past, to increase. In particular, pet dogs have higher intelligence and adaptability than other animals, and thus there are an increasing number of cases where they reside and live along with humans in indoor areas.

For this reason, as the companion animal-related industry develops, interest in the health management of companion animals is gradually increasing in order to live a happy life along with healthy companion animals for a long time.

Conventionally, in order to diagnose the health of companion animals, people visit animal hospitals along with the companion animals, or perform the self-diagnosis of the companion animals in homes by means of self-diagnostic kits.

However, when a person visits an animal hospital along with a companion animal and uses a health checkup, problems arise in that the person suffers from the inconvenience of having to visit an animal hospital every time and high health checkup expenses are burdensome. In contrast, in the case of self-diagnosis using a self-diagnostic kit in a home, a problem arises in that the use of the self-diagnostic kit is inconvenient because most self-diagnostic kits are kits for diagnosing health using blood.

As one of the technologies for solving the above problems, Korean Patent No. 10-1582732 discloses a companion animal health care device.

FIGS. 1a and 1b are views showing the conventional companion animal health care device. The conventional companion animal health care device will be described with reference to the accompanying drawings FIGS. 1a and 1b. When the conventional companion animal health care device is used as a leash for a companion animal, calorie consumption based on walk distance can be checked, and a health state can be measured by measuring the body weight of the companion animal by means of a simple method.

The conventional companion animal health care device will be described in greater detail. The conventional companion animal health care device includes: a first grip portion 1 the width of one end of which is greater than that of the other end thereof; a second grip portion 2 the width of one end of which is greater than that of the other end thereof and which is coupled to the other end of the first grip portion 1; an attachment/detachment member which selectively attaches and detaches the other end of the first grip portion 1 and the other end of the second grip portion 2 to and from each other; a weight measurement belt 4 which is stored in an internal spaced extending from the first grip portion 1 through the attachment/detachment member to the second grip portion 2 and both ends of which are fastened to the insides of the first and second grip portions 1 and 2, respectively; a leash hole which is provided at one end of the first or second grip portion 1 or 2 and into which a leash for a companion animal is mounted; a weight measurement sensor which is provided inside the first and second grip portions 1 and 2 and which measures the tensile stress applied to the weight measurement belt 4; and a slip prevention pad 6 which is formed on first sides of the first and second grip portions 1 and 2. The conventional companion animal health care device is used in such a manner that a leash is taken out of the leash hole 5 and then a walk is taken or in such a manner that weight is measured by wrapping and raising a companion animal using the weight measurement belt 4 in the state in which the first grip portion 1 and the second grip portion 2 have been gripped.

However, the conventional technology has a problem in that it is difficult to raise a companion animal when the companion animal struggles when the companion animal is wrapped and raised using the weight measurement belt 4. Even when the companion animal is raised using the weight measurement belt 4, a problem arises in that it is difficult to accurately measure the weight of the companion animal in the state in which the companion animal is moving.

Furthermore, the conventional technology is intended merely to measure the weight of a companion animal, and thus a problem arises in that it is difficult to perform the self-diagnosis of the health of a companion animal because it is difficult to determine the weight of a companion animal based on the type of companion animal, for example, in the case of a pet dog, the weight of the pet dog based on the type of dog.

### RELATED ART DOCUMENTS

### Patent Documents

Korean Patent No. 10-1582732 (Dec. 29, 2015)

Korean Patent Application Publication No. 10-2015-0115028 (Oct. 14, 2015)

Korean Patent Application Publication No. 10-2016-0036801 (Apr. 5, 2016)

Korean Patent Application Publication No. 10-2003-0032088 (Apr. 26, 2003)

### DISCLOSURE

### Technical Problem

The present invention has been conceived to overcome the above-described problems, and an object of the present invention is to provide a smart defecation plate including a companion animal medical examination kit, in which a medical examination kit is provided on the top of a pad part, thereby enabling the health state of a companion animal to be diagnosed using a fluid that is introduced into the pad part.

Another object of the present invention is to provide a smart defecation plate including a companion animal medical examination kit, which can measure the weight information of a companion animal by detecting a variation in the weight of an upper plate, thereby enabling the degree of obesity of the companion animal to be examined.

### Technical Solution

In order to accomplish the above objects, according to an embodiment of the present invention, there is provided a smart defecation plate including a companion animal medical examination kit, the smart defecation plate including: a body the center of which is formed in a concave shape; an upper plate which is coupled to the top of the body, and in which defecation holes are formed in a grid pattern; a pad part which includes a waterproof pad and absorption pads attached to the waterproof pad and configured to absorb a fluid entering through the defecation holes and which is disposed between the body and the upper plate; and a medical examination kit which is disposed on the top of the pad part.

Furthermore, the absorption pads may include a plurality of pads, and may be attached to the top of the waterproof pad so that parts to which the absorption pads are not attached guide the fluid, having passed through the defecation holes, to the center of the pad part.

Furthermore, the medical examination kit may include: an absorption portion which absorbs the fluid; a guide portion which guides the fluid absorbed via the absorption portion; and a diagnosis unit which generates disease information by diagnosing whether or not a disease is present through the antigen-antibody reaction of the fluid guided via the guide portion.

Furthermore, the medical examination kit may further include a control unit which includes: a diagnosis result display member which transmits disease information diagnosed by the diagnosis unit; and a power supply member which supplies power.

Furthermore, the body may be provided with one or more load cells which measure the weight information of a companion animal by detecting a variation in the weight of the upper plate.

Furthermore, the control unit may further include a module communication member which transmits at least one of the disease information diagnosed by the diagnosis unit and the weight information of the companion animal measured by the load cells.

Furthermore, the smart defecation plate may further include a central server which includes: a central communication member which receives at least one of disease information and weight information from the module communication member; a storage member which stores standard companion animal information; and a processing member which generates prescription information by comparing the disease information with the standard companion animal information stored in the storage member when the disease information is received via the central communication member and which generates degree-of-obesity information by comparing the received weight information with the standard companion animal information stored in the storage member when the weight information is received via the central communication member. In this case, the central communication member transmits at least one of the prescription information and the degree-of-obesity information generated by the processing member.

Furthermore, a catch hook which protrudes upward from the center of the body may be formed on the body, and a stop member which is provided at the center of the bottom of the pad part and which is fitted over the catch hook may be formed on the pad part.

Furthermore, the pad part may include fastening bands which fasten the medical examination kit.

### Advantageous Effects

In accordance with the smart defecation plate including a companion animal medical examination kit according to an embodiment of the present invention, the medical examination kit is provided on the top of the pad part, and thus an effect arises in that the health state of a companion animal may be diagnosed using a fluid that is introduced into the pad part.

Furthermore, according to the present invention, during the defecation activity of a companion animal, the weight of the companion animal is measured based on a variation in the weight of the upper plate, and thus effects arise in that accurate weight may be measured and the degree of obesity of the companion animal may be examined based on the measured weight.

Moreover, according to the present invention, an effect arises in that at least one of the disease information, prescription information, and degree-of-obesity information of a companion animal diagnosed by the medical examination kit may be easily viewed via a user terminal.

### DESCRIPTION OF DRAWINGS

FIGS. 1a and 1b are views showing a conventional companion animal health care device;

FIGS. 2a and 2b are views showing a smart defecation plate including a companion animal medical examination kit according to the present invention;

FIG. 3 is a view showing the medical examination kit of a smart defecation plate including a companion animal medical examination kit according to the present invention;

FIG. 4 is a view showing the control unit of the smart defecation plate including a companion animal medical examination kit according to the present invention;

FIG. 5 is a view showing states in which the smart defecation plate including a companion animal medical examination kit according to the present invention is practiced;

FIG. 6 is a view showing the central server of the smart defecation plate including a companion animal medical examination kit according to the present invention;

FIG. 7 is a view showing the catch hook and stop member of the smart defecation plate including a companion animal medical examination kit according to the present invention; and

FIG. 8 is a view showing the fastening bands of the smart defecation plate including a companion animal medical examination kit according to the present invention.

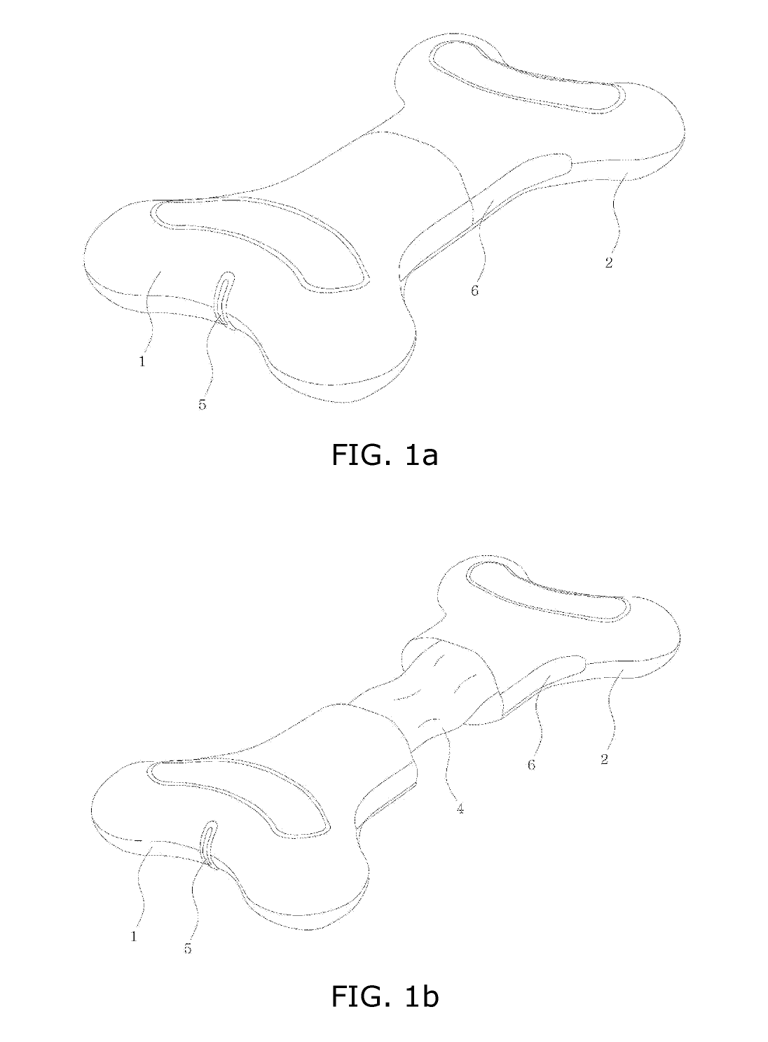

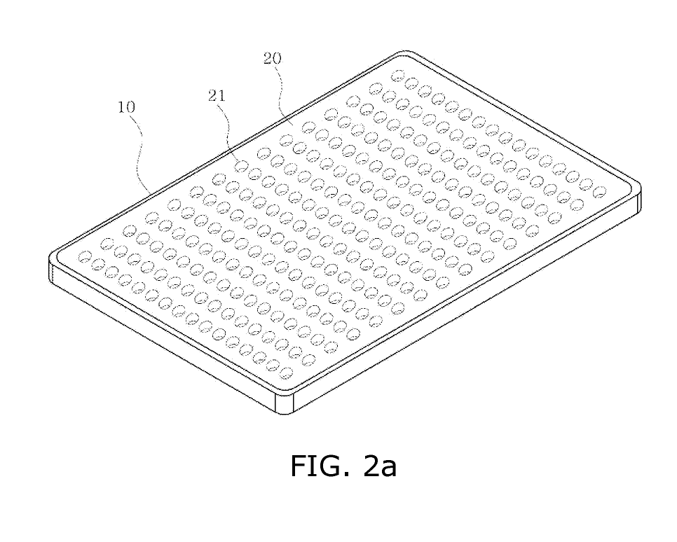

### BEST MODE

The present invention is subject to various modifications, and may have various embodiments. Specific embodiments will be illustrated in the drawings, and will be described in detail below. However, this is not intended to limit the present invention to the specific embodiments, but it should be understood that all modifications, equivalents and substitutions included in the spirit and technical scope of the present invention are included in the present invention.

When any component is described as being “connected” or “coupled” to another component, it should be interpreted that the former component may be directly connected or coupled to the other component but a third component may be present between the components.

In contrast, when any component is described as being directly “connected” or “coupled” to another component, it should be interpreted that a third component is not present between the components.

The terms used herein are used merely to describe specific embodiments, and are not intended to limit the present invention. Each singular expression used herein may include a plural expression unless clearly defined otherwise. The term “include” or “have” used herein is intended to specify a specific feature, number, process, operation, component, part or a combination thereof, but is not intended to exclude the presence or addition of a different feature, number, process, operation, part, or combination thereof.

Unless defined to the contrary, all terms, including technical terms and scientific terms, used herein have the same meanings as commonly understood by those having ordinary knowledge in the art to which the present invention pertains. The terms defined in commonly used dictionaries should be interpreted as having meanings consistent with relevant art documents and the present disclosure, and should not be interpreted in an ideal or overly formal sense unless expressly so defined herein.

The present invention will be described in greater detail below with reference to the accompanying drawings. Prior to the detailed description, it should be noted that the terms or words used in the present specification and the attached claims should not be interpreted as being limited to common or dictionary meanings and the technical terms used in the present specification of the present invention are used merely to describe specific embodiments based on the principle that an inventor may appropriately define the concepts of terms in order to describe his or her invention in the best way and are not intended to limit the present invention. Furthermore, the technical terms and scientific terms used have the meanings that are commonly understood by those having ordinary knowledge in the art to which the present invention pertains, unless defined to the contrary. In the following description and the accompanying drawings, a description of a well-known function or configuration that may unnecessarily make the gist of the present invention obscure will be omitted. The drawings that will be introduced below are provided as examples in order to fully convey the spirit of the present invention to those skilled in the art. Accordingly, the present invention is not limited to the drawings presented below, but may be embodied in different forms. Furthermore, throughout the specification, the same reference numerals designate the same components. It should be noted that the same components in the drawings are designated by the same reference symbols as much as possible.

The present invention relates to a smart defecation plate including a companion animal medical examination kit, in which a medical examination kit is provided on the top of a pad part, thereby enabling the health state of a companion animal to be diagnosed using a fluid that is introduced into the pad part.

The smart defecation plate including a companion animal medical examination kit according to the present invention will be described in detail below with reference to the accompanying drawings.

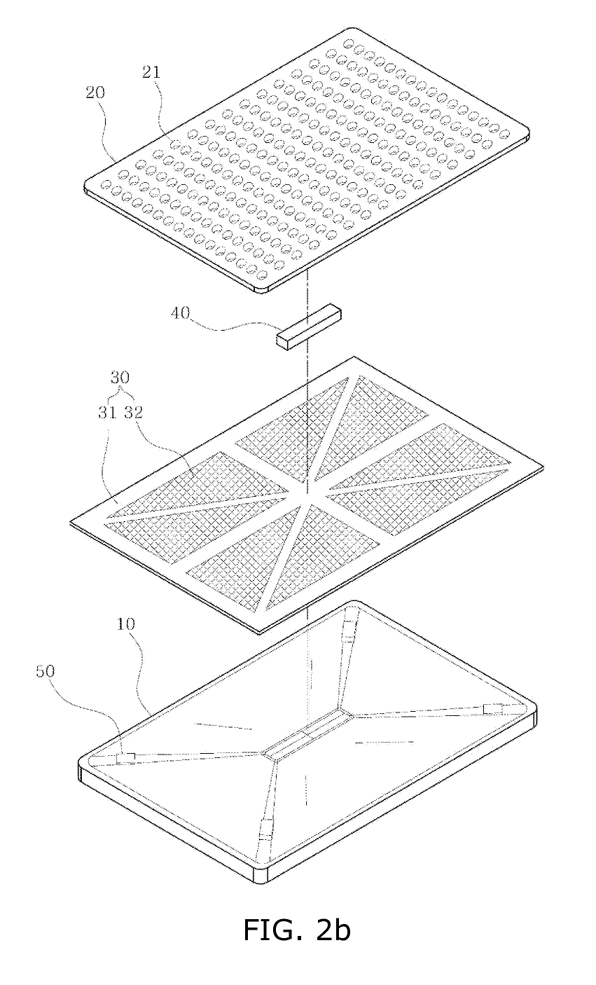

FIGS. 2a and 2b are views showing the smart defecation plate including a companion animal medical examination kit according to the present invention.

Referring to the accompanying FIGS. 2a and 2b, the smart defecation plate including a companion animal medical examination kit according to the present invention includes: a body 10 the center of which is formed in a concave shape; an upper plate 20 which is coupled to the top of the body 10, and in which defecation holes 21 are formed in a grid pattern; a pad part 30 which includes a waterproof pad 31 disposed between the body 10 and the upper plate 20 and absorption pads 32 attached to the waterproof pad 31 and configured to absorb a fluid entering through the defecation holes 21; and a medical examination kit 40 which is disposed on the top of the pad part 30.

In this case, the upper plate 20 is a component which is coupled by being fitted into a coupling groove formed along the periphery of the body 10. When the pad part 30 is disposed between the body 10 and the upper plate 20, the pad part 30 may be fastened in the state in which the center thereof has been pressed in a concave shape in accordance with the shape in which the center of the body 10 is concave.

Although the defecation holes 21 of the upper plate 20 are illustrated as being formed as a plurality of circular holes in the present invention, the circular holes may be modified into polygonal holes in an embodiment. The upper plate 20 may be modified into a common defecation plate form.

The absorption pads 32 of the pad part 30 include a plurality of pads, and are attached to the top of the waterproof pad 31 so that parts to which the absorption pads 32 are not attached guide a fluid, having passed through the defecation holes 21, to the center portion of the pad part 30.

In other words, the absorption pads 32 include a plurality of pads, and are attached to part of the top of the waterproof pad 31 so that parts to which the absorption pads 32 are not attached are connected to each other in a guide form such that a fluid, having passed through the defecation holes 21 of the upper plate 20, can flow to the center portion of the pad part 30 that is disposed in the state in which the center thereof has been pressed in a concave shape in accordance with the shape in which the center of the body 10 is concave.

Accordingly, when a companion animal passes urine onto the upper plate 20, the urine moves to the pad part 30 through the defecation holes 21 of the upper plate 20, a part of the urine moved to the pad part 30 is absorbed into the absorption pads 32 of the pad part 30, and another part of the urine not absorbed into the absorption pads 32 flows to the medical examination kit 40, disposed on the upper center portion of the pad part 30, along the waterproof pad 31 of the pad part 30.

The waterproof pad 31 of the pad part 30 may be made of a resin having a waterproof characteristic, such as a polyethylene film. The absorption pads 32 of the pad part 30 may include absorption layers made of the combination of one or more of a surface layer made of nonwoven material and pulp tissues, natural pulp, and a polymer absorber, or may be made of silica gel powder, paper, or cotton material.

Furthermore, the body 10 may be provided with one or more load cells 50 which measure the weight information of a companion animal by detecting a variation in the weight of the upper plate 20.

The load cells 50 are components which measure the weight information of a companion animal that steps on the upper plate 20. The load cells 50 measure the average weight of a companion animal by repeatedly measuring a variation in the weight of the upper plate 20, and generate the measured average weight as weight information.

Furthermore, the load cells 50 may measure the amount of urine and/or feces of a companion animal as the urine and/or feces of the companion animal are left on the upper plate 20 and the pad part 30 in the case where the companion animal that steps on the upper plate 20 passes urine and/or feces and then steps off the upper plate 20.

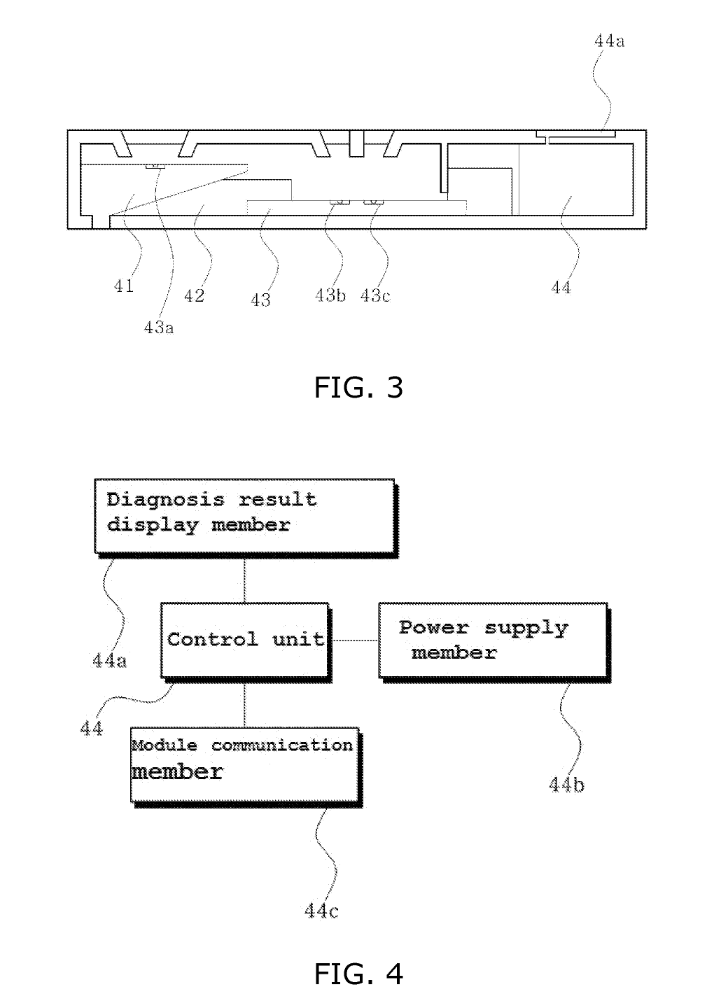

FIG. 3 is a view showing the medical examination kit of the smart defecation plate including a companion animal medical examination kit according to the present invention, and FIG. 4 is a view showing the control unit of the smart defecation plate including a companion animal medical examination kit according to the present invention.

Referring to the accompanying FIGS. 3 and 4, the medical examination kit 40 according to the present invention includes: an absorption portion 41 which absorbs a fluid; a guide portion 42 which guides the fluid absorbed via the absorption portion 41; and a diagnosis unit 43 which generates disease information by diagnosing whether or not a disease is present through the antigen-antibody reaction of the fluid guided via the guide portion 42. The housing of the medical examination kit 40 may be molded using polydimethylsiloxane (PDMS).

The medical examination kit 40 is a component which diagnoses the health of a companion animal by using a fluid that flows along the waterproof pad 31 of the pad part 30 as the pad part 30 is fastened in the state in which the center thereof has been pressed in a concave shape in accordance with the shape in which the center of the body 10 is concave. The medical examination kit 40 may be configured such that an adhesive portion is formed on the bottom thereof and is attached to the top of the pad part 30.

The absorption portion 41 is a component which is formed in the shape of a capillary tube having a size equal to or smaller than 0.5 mm, which is exposed out of the bottom of the medical examination kit 40 and which absorbs a fluid flowing along the waterproof pad 31. When a fluid flowing along the waterproof pad 31 of the pad part 30 is stagnated for a predetermined period in the state in which the medical examination kit 40 has been disposed on the top of the pad part 30, it is absorbed through the capillary tube phenomenon of the absorption portion 41.

In other words, a part of the urine of a companion animal is absorbed into the absorption portion 41 of the medical examination kit 40, and another part of the urine of the companion animal is absorbed into the absorption pads 32 of the pad part 30.

The diagnosis unit 43 may diagnose a disease based on the presence or absence of the positive or negative reaction of an antibody-bound conjugate band 43a, a test line band 43b, and a control line band 43c by using the antigen-antibody reaction of a fluid absorbed via the absorption portion 41 and guided by the guide portion 42, and may generate the disease information of the diagnosed disease.

The diagnosis unit 43 may diagnose the presence or absence of different diseases because it includes a plurality of bands exhibiting different reactions. For example, the diagnosis unit 43 may diagnose the presence or absence of diseases, for example, parvovirus, pancreatitis, and influenza.

In this case, the antibody-bound conjugate band 43a is formed by combining gold nano-particles with antibodies. The gold nano-particles refer to gold particles having a nanometer size, and refer to particles in the size range of 1 to 9 m. The antibody-bound conjugate band 43a diagnoses the presence or absence of a disease based on the fact that an antigen included in the urine of a companion animal takes on a specific color when it reacts with the gold nano-particle-bound antibody.

Furthermore, the control line band 43c takes on a specific color in order to check whether the gold nano-particle-bound antibodies have passed through the test line band 43b along with the urine of the companion animal, and determines whether the diagnosis of diseases has been correctly performed.

Furthermore, the diagnosis unit 43 may determine the health state of a companion animal by using the amount of urine and/or feces of the companion animal measured by the load cells 50.

The medical examination kit 40 further includes a control unit 44. The control unit 44 may include: a diagnosis result display member 44a which transmits disease information diagnosed by the diagnosis unit 43; a power supply member 44b which supplies power; and a module communication member 44c which transmits at least one of the disease information diagnosed by the diagnosis unit 43 and the weight information of a companion animal and the amount-of-urine and/or feces information of the companion animal measured by the load cells 50.

The diagnosis result display member 44a is a component which enables the presence or absence of a disease of a companion animal to be viewed when a user separates the upper plate 20 from the body 10 in order to replace the pad part 30. For example, the diagnosis result display member 44a displays the presence or absence of a disease of a companion animal via letters or an LED.

The module communication member 44c is a component which transmits at least one of the disease information of a companion animal, the weight information of the companion animal, and the amount-of-urine and/or feces information of the companion animal to, for example, the previously registered mobile phone terminal (not shown) of a user. The module communication member 44c may include a member using a communication network, such as a Bluetooth or Wi-Fi network, or the like.

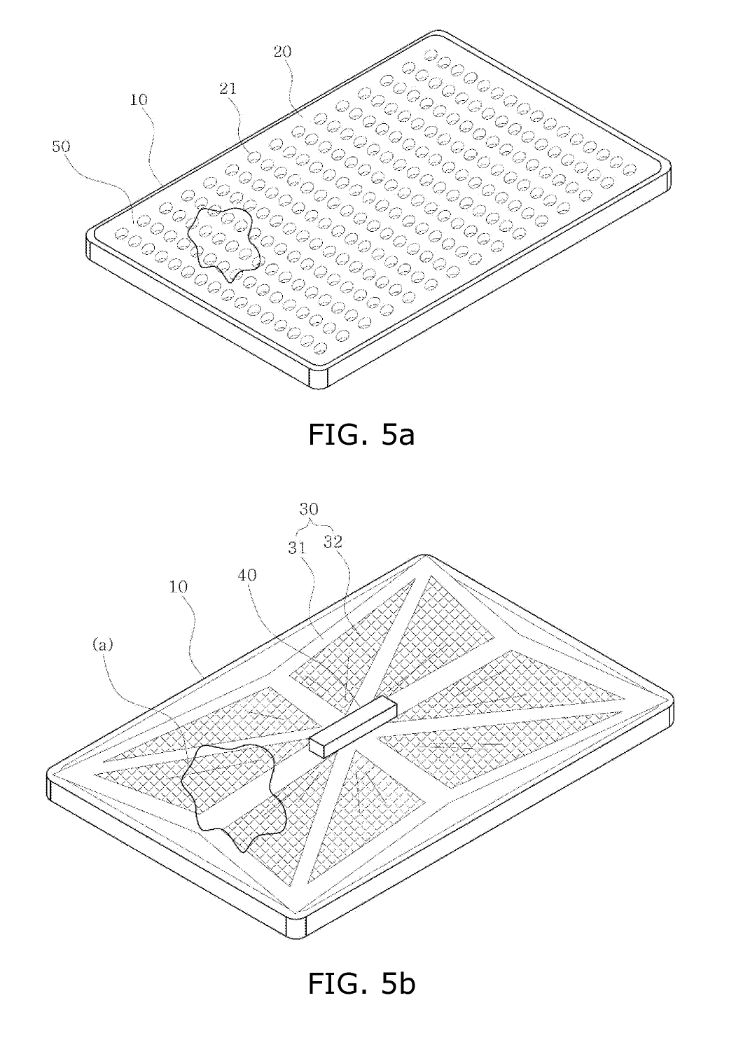

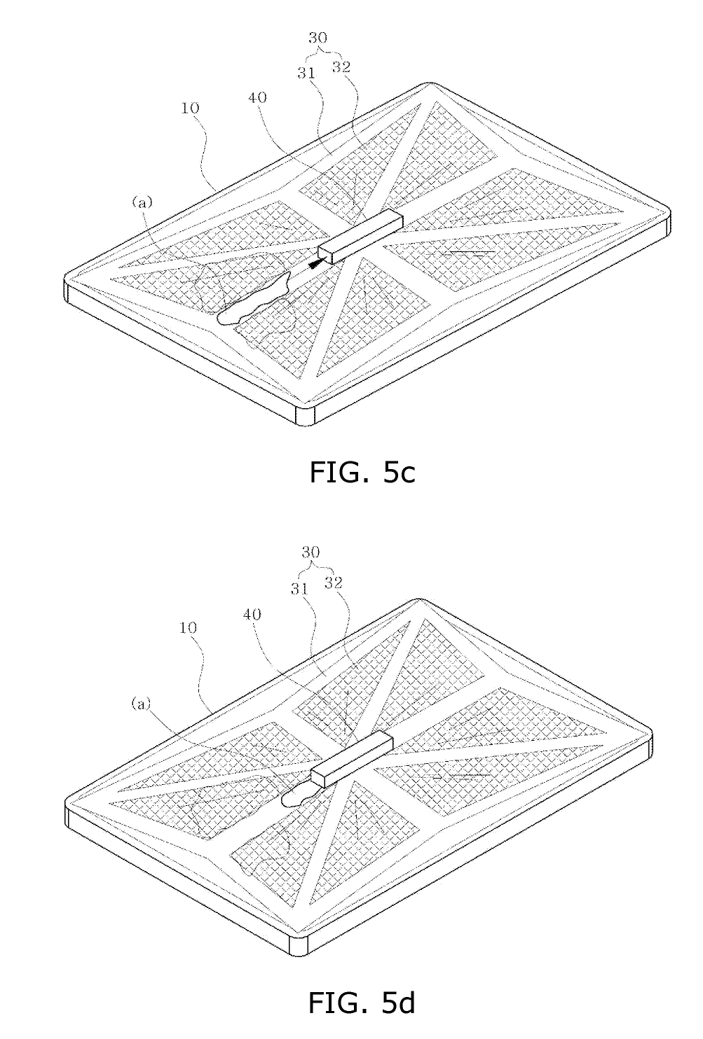

FIGS. 5a to 5d are views showing states in which the smart defecation plate including a companion animal medical examination kit according to the present invention is practiced.

Referring to the accompanying FIGS. 5a to 5d, FIG. 5a shows a state in which a companion animal has passed urine onto the upper plate 20 of the present invention, FIG. 5b shows a state in which the urine on the upper plate 20 has passed through the defecation holes 21 of the upper plate 20 and has moved to the pad part 30, FIG. 5c shows a state in which a part of the urine having moved to the pad part 30 has been absorbed to the absorption pads 32 of the pad part 30 and another part of the moved urine has not been absorbed to the waterproof pad 31 of the pad part 30, and FIG. 5d shows a state in which the urine not absorbed on the waterproof pad 31 moves to the center of the pad part 30 along the waterproof pad 31 of the pad part 30 that is fastened in the state in which the center thereof has been pressed in a concave shape in accordance with the shape in which the center of the body 10 is concave.

According to the present invention, the urine of the companion animal moved to the medical examination kit 40 through the process of FIGS. 5a to 5d may be diagnosed by the diagnosis unit 43 of the medical examination kit 40. The diagnosis unit 43 may generate disease information by using the results of the diagnosis of the urine.

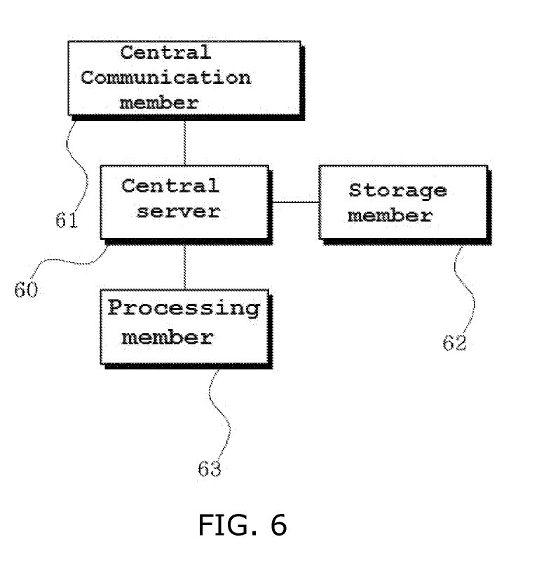

FIG. 6 is a view showing the central server of the smart defecation plate including a companion animal medical examination kit according to the present invention.

Referring to the accompanying FIG. 6, the smart defecation plate including a companion animal medical examination kit according to the present invention may further include a central server 60 including a central communication member 61, a storage member 62, and a processing member 63.

The central communication member 61 may receive at least one of disease information, weight information, and amount-of-urine and/or feces information from the module communication member 44c of the control unit 44.

The storage member 62 may store standard companion animal information and per-user companion animal history information.

The processing member 63 may generate prescription information by comparing disease information with the standard companion animal information stored in the storage member 62 when the disease information is received via the central communication member 61, and may generate degree-of-obesity information by comparing received weight information with the standard companion animal information stored in the storage member 62 when the weight information is received via the central communication member 61.

In this case, the central communication member 61 transmits at least one of the prescription information, the degree-of-obesity information, and the amount-of-urine and/or feces information generated by the processing member 63. The at least one of the prescription information, the degree-of-obesity information, and the amount-of-urine and/or feces information transmitted by the central communication member 61 may be transmitted to the previously registered mobile phone terminal of a user.

In other words, as the central server 60 manages the disease information, weight information, and amount-of-urine and/or feces information of a companion animal registered for each user and provides prescription information and degree-of-obesity information based on the disease information, the weight information, and the amount-of-urine and/or feces information to the user, the user may check the disease information, weight information and amount-of-urine and/or feces information of a companion animal, prescription information based on the disease information, and degree-of-obesity information based on the weight information without visiting a hospital.

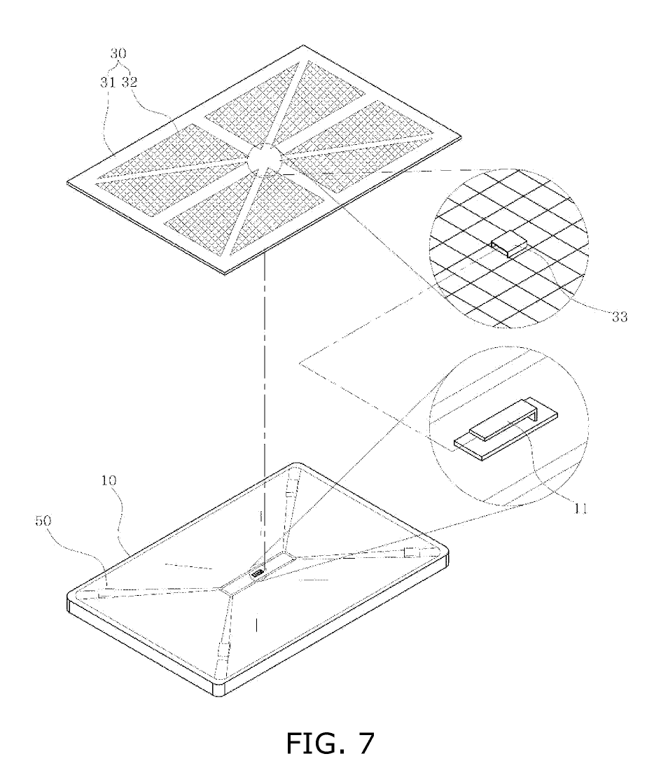

FIG. 7 is a view showing the catch hook and stop member of the smart defecation plate including a companion animal medical examination kit according to the present invention.

Referring to the accompanying FIG. 7, a catch hook 11 which protrudes upward from the center of the body 10 may be formed on the body 10 according to the present invention, and a stop member 33 which is provided at the center of the bottom of the pad part 30 and which is fitted over the catch hook 11 may be formed on the pad part 30 according to the present invention.

In other words, the defecation plate according to the present invention is configured such that the catch hook 11 of the body 10 is inserted into the stop member 33 of the pad part 30, thereby preventing the pad part 30 from being shaken on the body 10 or preventing the location of the pad part 30 from becoming unstable on the body 10.

Although the configurations of the catch hook 11 and the stop member 33 shown in FIG. 7 of the present invention are configured as a common hook and a common stop member, the catch hook 11 and the stop member 33 are intended to form a systematic relationship between the body 10 and the pad part 30, and thus it may possible to freely change the shapes under the same purpose.

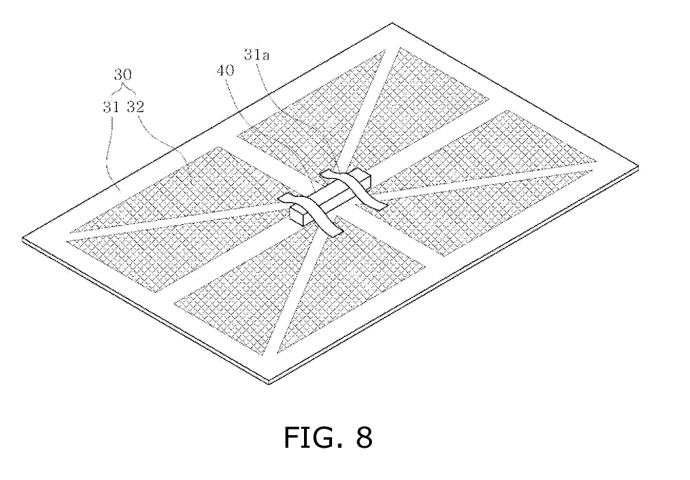

FIG. 8 is a view showing the fastening bands of the smart defecation plate including a companion animal medical examination kit according to the present invention.

Referring to the accompanying FIG. 8, the pad part 30 according to the present invention may further include fastening bands 31a which fasten the medical examination kit 40.

The fastening bands 31a may be made of elastic material. The fastening bands 31a enable the medical examination kit 40 to be fastened to the pad part 30 by inserting the medical examination kit 40 under the fastening bands 31a in the state in which the center portions of the fastening bands 31a have been spaced apart from the pad part 30.

These fastening bands 31a may be formed on, for example, only the portions to which the absorption pads 32 are not attached. In an embodiment, the fastening bands 31a may be freely modified into pocket shapes, other than band shapes.

According to the above-described present invention, the medical examination kit is provided on the top of the pad part, and thus an effect arises in that the health state of a companion animal may be diagnosed using a fluid that is introduced into the pad part. Furthermore, during the defecation activity of a companion animal, the weight of the companion animal is measured based on a variation in the weight of the upper plate, and thus effects arise in that accurate weight may be measured and the degree of obesity of the companion animal may be examined based on the measured weight.

While the foregoing description has been given by presenting the various embodiments of the present invention, the present invention is not necessarily limited to these embodiments. It will be understood by those having ordinary knowledge in the art to which the present invention pertains that various substitutions, modifications, and alterations may be made without departing from the technical spirit of the present invention.

1. A smart defecation plate including a companion animal medical examination kit, the smart defecation plate comprising:
a body (10) a center of which is formed in a concave shape;
an upper plate (20) which is coupled to a top of the body (10), and in which defecation holes (21) are formed in a grid pattern;
a pad part (30) which includes a waterproof pad (31) and absorption pads (32) attached to the waterproof pad (31) and configured to absorb a fluid entering through the defecation holes (21) and which is disposed between the body (10) and the upper plate (20); and
a medical examination kit (40) which is disposed on a top of the pad part (30).

2. The smart defecation plate of claim 1, wherein the absorption pads (32) comprise a plurality of pads, and are attached to a top of the waterproof pad (31) so that parts to which the absorption pads (32) are not attached guide the fluid, having passed through the defecation holes (21), to a center of the pad part (30).

3. The smart defecation plate of claim 1, wherein the medical examination kit (40) comprises: an absorption portion (41) which absorbs a fluid; a guide portion (42) which guides the fluid absorbed via the absorption portion (41); and a diagnosis unit (43) which generates disease information by diagnosing whether or not a disease is present through an antigen-antibody reaction of the fluid guided via the guide portion (42).

4. The smart defecation plate of claim 3, wherein the medical examination kit (40) further comprises a control unit (44) which comprises: a diagnosis result display member (44a) which transmits disease information diagnosed by the diagnosis unit (43); and a power supply member (44b) which supplies power.

5. The smart defecation plate of claim 4, wherein the body (10) is provided with one or more load cells (50) which measure weight information of a companion animal by detecting a variation in weight of the upper plate (20).

6. The smart defecation plate of claim 5, wherein the control unit (44) further comprises a module communication member (44c) which transmits at least one of the disease information diagnosed by the diagnosis unit (43) and the weight information of the companion animal measured by the load cells (50).

7. The smart defecation plate of claim 6, further comprising a central server (60) which comprises:
a central communication member (61) which receives at least one of disease information and weight information from the module communication member (44c);
a storage member (62) which stores standard companion animal information; and
a processing member (63) which generates prescription information by comparing the disease information with the standard companion animal information stored in the storage member (62) when the disease information is received via the central communication member (61) and which generates degree-of-obesity information by comparing the received weight information with the standard companion animal information stored in the storage member (62) when the weight information is received via the central communication member (61);
wherein the central communication member (61) transmits at least one of the prescription information and the degree-of-obesity information generated by the processing member (63).

8. The smart defecation plate of claim 1, wherein:
a catch hook (11) which protrudes upward from a center of the body (10) is formed on the body (10); and
a stop member (33) which is provided at a center of a bottom of the pad part (30) and which is fitted over the catch hook (11) is formed on the pad part (30).

9. The smart defecation plate of claim 1, wherein the pad part (30) comprises fastening bands (31a) which fasten the medical examination kit (40).
